(19) United States
(12) Patent Application Publication
(10) Pub. No.: US 2014/0088520 A1
(43) Pub. Date: Mar. 27, 2014

(54) MICRO TOOTHED CAP DEVICE WITH A CAVITY TO PENETRATE THROUGH A HAIR WEAVE TO THE SCALP

(71) Applicants: Annalica Michele Kong, London (GB); Josiah Andrew Jackman, Port of Spain (TT)

(72) Inventors: Annalica Michele Kong, London (GB); Josiah Andrew Jackman, Port of Spain (TT)

(21) Appl. No.: 13/629,571

(22) Filed: Sep. 27, 2012

(51) Int. Cl. A61M 35/00; A61D 7/00

(52) U.S. Cl. 604/290; 604/311

(57) ABSTRACT

A micro toothed cap device with a cavity can penetrate through a hair weave to exfoliate and stimulate the scalp. The device can be used to apply medicated products directly into the hard-to-reach areas under a hair weave to easily access the scalp. The device is small enough to penetrate to the undergrowth under the plaits, cornrows and wefts, allowing the scalp to be exfoliated, cleansed and treated properly without shifting the weft out of place.

### BACKGROUND OF THE INVENTION

The present invention relates to hair and scalp accessories and, more particularly, to a device for penetrating through a hair weave to apply medicated product or air to the scalp.

There are products for the scalp, but no device to get these products to difficult to reach area, for example, areas under a hair weave.

As can be seen, there is a need for a device that can be used to apply medicated products to the scalp, including the hard to reach areas, such as under a hair weave.

### SUMMARY OF THE INVENTION

In one aspect of the present invention, an applicator device comprises a main body having a flexible, compressible housing; a detachable syringe disposed on a first end of the main body; and a plurality of teeth, arranged in a row or rows, attached to the main body, the plurality of teeth each having applicator holes formed at sides thereof proximate to ends of the plurality of teeth.

In another aspect of the present invention, an applicator device comprises a main body having a flexible, compressible housing; a detachable syringe disposed on a first end of the main body; a syringe applicator hole disposed at an end of the detachable syringe; a plurality of teeth, arranged in a row or rows, attached to the main body; and applicator holes formed at sides of each of the plurality of teeth proximate to ends of the plurality of teeth.

In a further aspect of the present invention, a method for applying a treatment to the scalp under a hair weave comprises placing a plurality of teeth extending from a main body of a treatment application device under the hair weave to the scalp; and squeezing the main body to force medicated treatment out of the main body, through the teeth and out through holes at an end of each of the plurality of teeth.

These and other features, aspects and advantages of the present invention will become better understood with reference to the following drawings, description and claims.

### BRIEF DESCRIPTION OF THE DRAWINGS

FIG. 1 is a perspective view of a toothed cap device with a cavity to penetrate through a hair weave according to an exemplary embodiment of the present invention;

FIG. 2 is a side view of the toothed cap device of FIG. 1;

FIG. 3 is a cross-sectional view taken along line 3-3 of FIG. 2;

FIG. 4 is a cross-sectional view showing liquid remedies/solution flowing through a detachable syringe of the toothed cap device of FIG. 1;

FIG. 5 is a detailed top view of a detachable toothed cap end of the toothed cap device of FIG. 1; and

FIG. 6 is a cross-sectional view taken along line 6-6 of FIG. 5 showing liquid remedies/solution flowing through a detachable toothed cap end of the toothed cap device of FIG. 1.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a micro toothed cap device with a cavity to penetrate through a hair weave to exfoliate and stimulate the scalp. The device can be used to apply medicated products directly into the hard-to-reach areas under a hair weave to easily access the scalp. The device is small enough to penetrate to the undergrowth under the plaits, cornrows and wefts, allowing the scalp to be exfoliated, cleansed and treated properly without shifting the weft out of place.

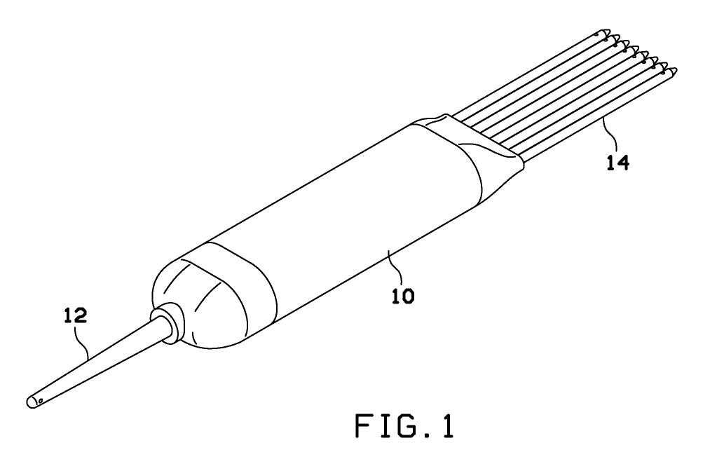

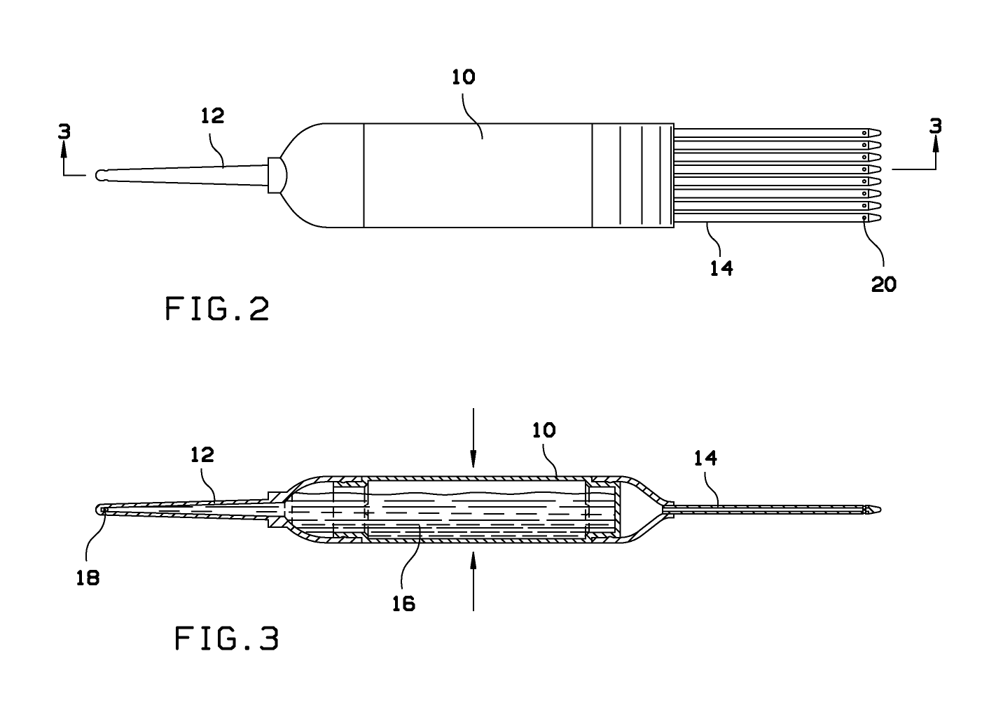

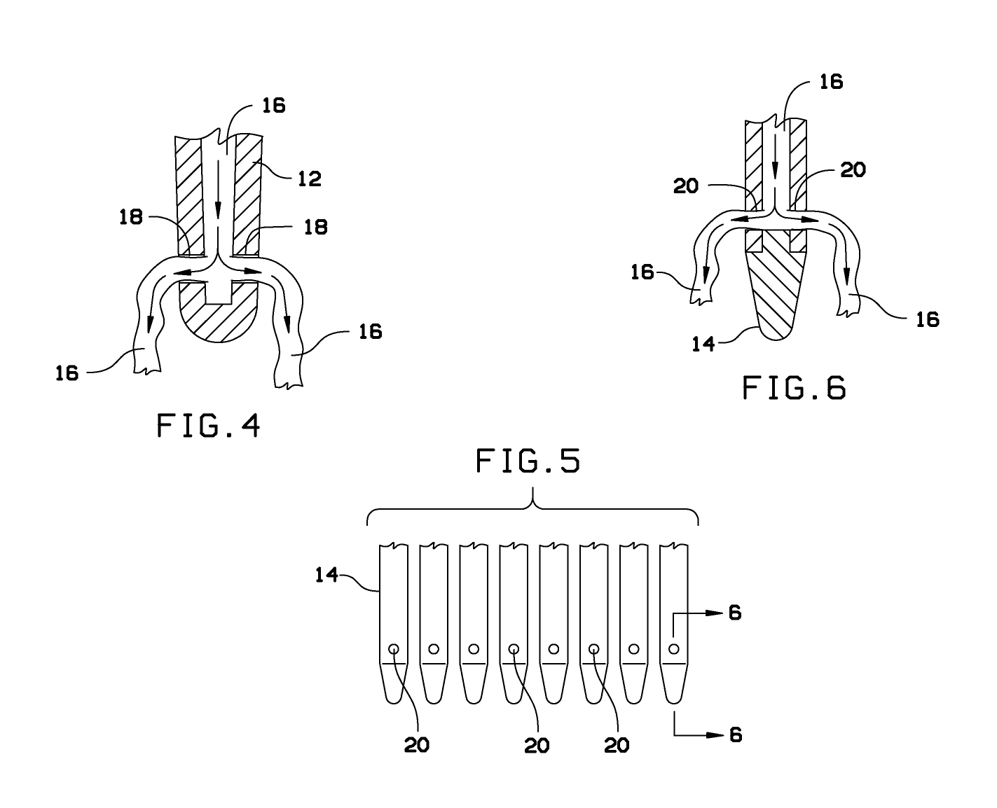

Referring now to FIGS. 1 through 6, a hair product transporter/micro toothed cap device can include a main body 10 having a detachable syringe 12 disposed on one side thereof and a toothed cap end having a plurality of teeth 14 disposed on an opposite end thereof. The detachable syringe 12 can include an applicator hole 18 to allow liquid remedies/solution 16 to flow therefrom.

The toothed cap end can include a plurality of teeth 14, typically from about 2 to 12 teeth, often about 8 teeth. The teeth can be hollow to communicate with the main body 10 so that fluids can be expelled from the main body 10, through the teeth and out through application holes 20 at the end of each of the plurality of the teeth. The application holes 20 are disposed on sides of the teeth 14, proximate to ends of the teeth 14. The teeth 14 can be about 40 to 100 mm in width and about 2 to 5 mm in diameter, for example.

The teeth 14 can be used to transport hair product smoothly into the undergrowth of a weave when the device is moved under pressure of the fingers to compress the main body 10 and expel the liquid out of the teeth 14.

The main body 10 can be refilled through an opening at the top thereof when the syringe 12 is removed. In some embodiments, the syringe 12 can be used to pull product into the main body 10. The main body 10 can be made from a flexible and durable material, such as low density polyethylene or rubber, for example. The teeth 14 can be made from various materials, such as acrylonitrile, butadiene, styrene, silicone or stainless steel, for example. The tips of the teeth 14 can be tapered to a blunt, closed-off tip.

As described above, the device of the present invention is configured and designed to allow a hair weave wearer to penetrate through a hair weave to the scalp. The device can be used for home administration of products to the scalp or can be used by trychologists to treat medical scalp problems. A veterinarian can use the device of the present invention to treat animals with fur to administer remedies for medical problems on the skin.

The main body can be pre-filled with medication or can be provided empty for the user to fill as desired. While the above describes using a medication in the device of the present invention, the holes can function to deliver not only liquids, but also air in an exfoliation process.

It should be understood, of course, that the foregoing relates to exemplary embodiments of the invention and that modifications may be made without departing from the spirit and scope of the invention as set forth in the following claims.

1. An applicator device comprising:
a main body having a flexible, compressible housing;
a detachable syringe disposed on a first end of the main body; and
a plurality of teeth, arranged in a row, attached to the main body, the plurality of teeth each having applicator holes formed at sides thereof, the applicator holes disposed only proximate to ends of the plurality of teeth.

2. The applicator device of claim 1, further comprising a syringe applicator hole disposed at an end of the detachable syringe.

3. The applicator device of claim 1, wherein liquid remedies/solution is operable to pass from the main body, through the teeth and out through the applicator holes when the main body is squeezed.

4. The application device of claim 1, wherein from 6 to 10 teeth are arranged in the row about ¼ inch in height.

5. An applicator device comprising:
a main body having a flexible, compressible housing;
a detachable syringe disposed on a first end of the main body;
a syringe applicator hole disposed at an end of the detachable syringe;
a plurality of teeth, arranged in a row, attached to the main body; and
applicator holes formed at sides of each of the plurality of teeth, the applicator holes disposed only proximate to ends of the plurality of teeth.

6. A method for applying a treatment to the scalp under a hair weave, comprising:
placing a plurality of teeth extending from a main body of a treatment application device under the hair weave to the scalp; and
squeezing the main body to force medicated treatment out of the main body, through the teeth and out through holes disposed only at an end of each of the plurality of teeth.

7. The method of claim 6, further comprising filling the main body through a detachable syringe at one end of the main body.

8. The method of claim 6, wherein the plurality of teeth are aligned in a single row.

9. The method of claim 8, wherein the single row includes from 6 to 10 teeth.
